(19) United States
(12) Patent Application Publication
(10) Pub. No.: US 2004/0018606 A1
(43) Pub. Date: Jan. 29, 2004

(54) Control of protein systemic delivery by hypoxia using a tet-HIF1-alpha chimeric transactivator

(76) Inventors: Delphine Bohl, Palaiseau (FR); Michael Heard, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/425,833

(22) Filed: Apr. 30, 2003

Related U.S. Application Data
(60) Provisional application No. 60/376,269, filed on Apr. 30, 2002.

(51) Int. Cl. C12Q 1/68; C07H 21/04; C12N 9/22; C12P 21/02; C12N 5/06

(52) U.S. Cl. 435/199; 435/6; 435/69.1; 435/320.1; 435/325; 536/23.2

(57) ABSTRACT

An isolated polynucleotide which codes for a domain of a transcription factor, where the domain confers to the transcription factor susceptibility to degredation under normoxia conditions. Also provided is a chimeric transactivator containing such a domain and to polynucleotides and vectors which code for the same. A method of expressing a target gene in a subject invloving the use of such a chimeric transactivator is also provided. In particular, such a method can be used to increase the number of red blood cells or blood vessels in the subject.

### CONTINUING APPLICATION INFORMATION

This application claims benefit of U.S. provisional application serial No. 60/376,269, filed on Apr. 30, 2002, and incorporated herein by reference.

### FIELD OF THE INVENTION

The present invention relates to an isolated polynucleotide which codes for a domain of a transcription factor, where the domain confers to the transcription factor susceptibility to degredation under normoxia conditions. The present invention also relates to a chimeric transactivator containing such a domain and to polynucleotides and vectors which code for the same. The present invention also relates to a method of expressing a target gene in a subject invloving the use of such a chimeric transactivator. In particular, such a method can be used to increase the number of red blood cells or blood vessels in the subject.

### BACKGROUND OF THE INVENTION

Erythropoietin (EPO) is a glycoprotein hormone secreted by the kidney and in some situations by the liver (1). Its main role is to induce terminal differentiation of erythroid precursors into red blood cells (2, 3). Increased secretion of Epo in kidney or liver tumors induced polycythemia, while in chronic renal failure reduced secretion of Epo induces severe anemia. Recombinant human erythropoietin (rHuEpo) was the first hematopoietic growth factor produced by genetic engineering. rHuEpo is administered to patients with chronic renal failure. It induces erythropoietic activity with an increase in reticulocyte cell counts and in hemoglobin concentration. Extension of rHuEpo applications to other pathologies such as anemia associated with AIDS or cancer, anemia from inflammatory origin, self-transfusions, and also to hemoglobinopathies, is limited by cost-effectiveness. The future of Epo for those patients implicates either a reduction of the production costs or the development of alternative methods of administration. Epo secretion from genetically modified tissues could play a role in this context. It is thought that a gene therapy protocol may be proposed to patients only if gene transfer is performed as a low cost, low risk, and simple protocol, allowing long-term secretion of the protein at controlled levels.

Ideally, the delivery of adequate amounts of Epo for a replacement therapy in Epo-dependent anemias would reproduce the physiological regulation. The physiological inducer of Epo secretion from kidney tubular cells is hypoxia. Sequences located 5′ and 3′ of the Epo gene are well conserved from mouse to human. In the 5′ end a region of 400 bp of the promoter was identified but seemed to play a secondary role in the hypoxic regulation (4). At the 3′ end of the gene a short sequence (5′-TACGTGCT-3′) was identified as the most important sequence implicated in the response to hypoxia (5, 6). This sequence binds under hypoxic conditions a transcription factor named HIF-1 (hypoxia-induced factor). Hypoxic-inducible expression of HIF-1 was detected in many cellular cell lines which do not express the Epo gene (8, 9) and was involved in the hypoxic regulation of many other genes, suggesting a important and wide role of HIF-1 in oxygen regulation (10, 11). A first attempt to control Epo secretion in response to hypoxia was reported using the phosphoglycerate kinase promoter, which contains HIF-1 binding sites (12). Hypoxic regulation was studied on a period of 24 hours only and induction levels were barely significant.

The two subunits of HIF-1, HIF-1α and HIF-1β, have been cloned (13). HIF-1β was identified as the RNT1 factor, a protein implicated in the removal of toxic products from cells. On the contrary HIF1α is highly specific of the hypoxic response.

The structure of HIF1-α was studied in details. In the N-terminal part of the protein, a motive called bHLH is implicated in the dimerization of the protein and the specific binding to DNA. In the C-terminal part of the protein two important domains are implicated in the transactivation process: one TAD-N domain between amino acids 531 and 575 and one TAD-C domain between amino acids 813 and 826 (14, 15, 16). A domain responsible for the degradation of the protein under normoxia is localized between amino acids 401 and 603 (17, 18). In one study authors identified a short sequence of 15 amino acids (557-571) which seemed crucial in the stabilization of the protein under hypoxia (19). Foreign proteins on which these different regulatory domains were transferred, appeared degraded in an oxygen-dependent fashion in transient transfection experiments (15, 18, 20).

It was shown recently that the activation mechanism of HIF1 implicated an oxygen- and iron-dependa t hydroxylase activity (21, 22). An enzyme called HIF1-α prolyl hydroxylase could be the oxygen sensor. Under normoxia, mRNA levels are stable, HIF-1α and ARNT1 are stable. AT the protein levels, whereas ARNT1 levels are stable, HIF-1α protein levels are increased under hypoxia. It was shown that HIF1-α binds to the pVHL factor (Von Hippel-Lindau) after hydroxylation. The pVHL protein belongs to a complex with an E3 ubiquitin ligase activity. So, HIF1-α is ubiquitinylated under normoxia and degradated by the proteasome (13, 20, 23). Under hypoxia or in the presence of cobaltous chloride or desferioxamine, HIF-1α is not hydroxylated and can't bind to pVHL. The hypoxic signal than activates the nuclear translocation of the protein, which binds to his co-factor ARNT1.

We previously described various gene transfer vectors that can induce a long-term and tightly regulated secretion of Epo from skeletal muscles in response to tetracycline (24-26). We have modified this regulatory system (originally described by the group of Hermann Bujard (27) for responsiveness to pO2. Various fragments of the transcription factor HIF1-alpha were fused to the tetracycline transactivator protein tTa. We show that these chimeric transactivators were degradated by the proteasome under normoxia, and that they stimulated transcription under hypoxia in vitro. In vivo after gene transfer with AAV-2 vectors in mice, we show that the expression of a foreign protein could be regulated in rsponse to a physiological stimulus like the partial pressure in oxygen.

Two publications of relevance are WO 01/36313,: Nucleic Acid Construct Bearing a System Regulating the Expression of a Gene and Payen et al., J. Gene Med., 2001, 3: 498-504.

In the paper by Payen et al. authors introduced into the transactivator tTA a domain of HIF1-α corresponding to amino acids between 531 and 634. In vitro experiments results shows that under normoxia this domain reduced the background level of transactivation of the tetO-CMV promoter. The mechanism implicated in this regulation was not analyzed. Nothing proves that the chimeric protein is degradated by the proteasome. Payen et al. don't analyze the mechanism implicated in the instability of the chimeric transactivator under normoxia. Several mechanisms independent of the proteasome degradation could be implicated. The motive of HIF1-α was not responsible for the transactivation under hypoxia. In real, induction was obtained only with HRE elements of the PGK promoter placed upstream and downstream of the transactivator transcriptional unit. In the system described by Payen et al. two elements are necessary: one domain of HIF1-α in the tTA protein for the degradation activity under normoxia and HRE sequences upstream and/or downstream of the tTA transcriptional unit for the induction under hypoxia.

Payen et al. don't give the proof that the regulation in vivo was related to the partial oxygen pressure. In in vivo experiment they don't make any real demonstration of the hypoxid regulation. Their model implicates an hypoxid state into Lewis lung carcinoma. Plasmids were transferred into tumors and mice were sacrificed 2 days later. Only 3 mice out of 9 have a higher level of expression in the tumor. However nothing proves that this is the hypoxic state of cells that is responsible. Other environmental parameters could have influenced the expression of the reporter gene into tumors.

### SUMMARY OF THE INVENTION

The present inventors previously described various gene transfer vectors that can induce a long-term and tightly regulated secretion of Epo from skeletal muscles in response to tetracycline (17-19). The present inventors have modified this regulatory system (originally described by the group of Hermann Bujard (20) for responsiveness to pO2). Various fragments of the transcription factor HIF1-alpha were fused to the tetracycline transactivator protein tTA. Herein it is shown that these chimeric transactivators were degradated by the proteasome under normoxia, and that they stimulated transcription under hypoxia in vitro. In vivo after gene transfer with AAV-2 vectors in mice, it is shown that the expression of a foreign protein could be regulated in response to a physiological stimulus like the partial pressure of oxygen.

Thus, the present invention relates to an isolated polynucleotide which codes for a domain of a transcription factor, wherein the domain confers to the transcription factor susceptibility to degradation under normoxia conditions.

In one embodiment, the polynucleotide codes for a domain of HIF1-alpha.

In another embodiment, the domain comprises amino acids between residues 530 to 603 of the HIF1-alpha.

In preferred embodiments, the domain comprises amino acids between residues 530 to 658 or 601 to 658 or part thereof comprising at least amino acids 530 to 603 of the HIF1-alpha.

In another embodiment, the domain comprises amino acids 530 to 603 of the HIF1-alpha.

In preferred embodiments, the domain comprises amino acids between residues 530 to 658 or 601 to 658 or part thereof comprising at least amino acids 530 to 603 of the HIF1-alpha.

In another embodiment, the domain is amino acids 530 to 603 of the HIF1-alpha.

In preferred embodiments, the domain is amino acids 530 to 658 or 601 to 658 or part thereof comprising at least amino acids 530 to 603 of the HIF1-alpha.

The present invention also relates to a chimeric transactivator comprising a domain of a transcription factor, wherein the domain confers to the transcription factor susceptibility to degredation under normoxia conditions.

In one embodiment, the transactivator comprises the tetracycline transactivator protein.

In another embodiment, the transcription factor is HIF1-alpha.

In another embodiment, the domain comprises amino acids between residues 530 to 603 of the HIF1-alpha.

In another embodiment, the domain comprises amino acids 530 to 603 of the HIF1-alpha.

In another embodiment, the domain is amino acids 530 to 603 of the HIF1-alpha.

In preferred embodiments, the domain is amino acids 530 to 658 or 601 to 658 or part thereof comprising at least amino acids 530 to 603 of the HIF1-alpha.

The present invention also relates to an isolated polynucleotide which codes for the chimeric transactivator described above.

In one embodiment, the chimeric transactivator comprises the tetracycline transactivator protein.

In another embodiment, the transcription factor is HIF1-alpha.

In another embodiment, the domain comprises amino acids between residues 530 to 603 of the HIF1-alpha.

In another embodiment, the domain comprises amino acids 530 to 603 of the HIF1-alpha.

In preferred embodiments, the domain is amino acids 530 to 658 or 601 to 658 or part thereof comprising at least amino acids 530 to 603 of the HIF1-alpha.

The present invention also relates to a vector comprising the polynucleotide described above.

In one embodiment, the chimeric transactivator comprises the tetracycline transactivator protein.

In another embodiment, the transcription factor is HIF1-alpha.

In another embodiment, the domain comprises amino acids between residues 530 to 603 of the HIF1-alpha.

In another embodiment, the domain comprises amino acids 530 to 603 of the HIF1-alpha.

In preferred embodiments, the domain is amino acids 530 to 658 or 601 to 658 or part thereof comprising at least amino acids 530 to 603 of the HIF1-alpha.

The present invention also provides a composition, comprising:

(a) the polynucleotide described above and

(b) a polynucleotide which contains a sequence that codes for a target gene and a promoter which is regulated by the chimeric tranactivator coded for by (a).

In one embodiment, the chimeric transactivator comprises the tetracycline transactivator protein.

In another embodiment, the transcription factor is HIF1-alpha.

In another embodiment, the domain comprises amino acids between residues 530 to 603 of the HIF1-alpha.

In another embodiment, the domain comprises amino acids 530 to 603 of the HIF1-alpha.

In another embodiment, the domain is amino acids 530 to 658 or 601 to 658 or part thereof comprising at least amino acids 530 to 603 of the HIF1-alpha.

In another embodiment, the target gene is erythropoietin.

In another embodiment, the target gene is VEGF.

The present invention also relates to an isolated polynucleotide which hybridizes under stringent conditions to the sequence complimentary to the polynucleotide which codes for the HIF1-alpha domain of the invention, provided said isolated polynucleotide codes for a polynucleotide which is involved in the the degredation by the proteosome under normoxia and transactivation of a given promoter under hypoxia. Stringent conditions are, for example:

Prehybridization solution: 20×SSPE (3.6 M NaCl, 0.2 sodium phosphate, 0.02M EDTA: 100× Denhards solution (2% BSA, 2% Ficoll, 2% PVP); 10% SDS

Add 10 mg of salmon sperm DNA (pre-heated to 100 deg C. for 5 minutes).

Prehybridize for 1 h at 65 deg C.

Add denatured probe (pre-heated to 100 deg C. for 5 minutes) and incubate at 65 deg C. for 12 h.

The present invnetion also relates to a method of expressing a target gene in a subject, comprising administering the composition described above to the subject.

In one embodiment, the subject is a mammal.

In another embodiment, the subject is a human.

In one embodiment, the target gene is erythropoietin.

In another embodiment, the target gene is VEGF.

The present invention also provides a method of increasing the number of blood vessels in a subject, comprising administering an effective amount of the composition of described above to the subject.

In one embodiment, the subject is a mammal.

In another embodiment, the subject is a human.

A more complete appreciation of the invention and many of the attendant advantages thereof will be readily obtained as the same becomes better understood by reference to the following Figures in conjunction with the detailed description below.

### BRIEF DESCRIPTION OF THE FIGURES

FIG. 1. Structure of a transactivator sensitive to pO2.

FIG. 2. Regulation of the transcriptional activity of THV fusion proteins in HeLa-16 cells under normoxia. HeLa-16 cells were electroporated with plasmids: pTV, pTHV 401-603, pTHV 401-658, pTHV 530-603, pTHV 530-658 or pTHV 530-826. Cells were then kept 24 hours at 21% O2 and treated in the presence or the absence of doxyxycline (1 μg/ml). Luciferase activity was measured. One representative experiment out of three is shown.

FIG. 3. Hypoxic induction of the transcriptional activity of THV proteins in HeLa-16 cells under different hypoxic conditions. HeLa-16 cells were electroporated with the different plasmids as in FIG. 1. Cells were then split in aliquots and either placed under 1% O2 or treated 24 hours with different chemicals: desferrioxamine (Def), cobaltous chloride (Co) or 2-2′-dipyridyl (DP). Cells were analysed for luciferase activities (FIGS. 3A and 3B). One representative experiment out of three is shown. Cell extracts were also performed for wertern blot analysis (FIG. 3C). Electroporated HeLa-16 cells were subjected to immunoblot analysis using anti-VP16 antibodies to determine THV protein levels. C: control cells electroporated with a control plasmid. N: normoxia 21% O2; H: hypoxia, 1% O2; DP: 2-2′-dipyridyl (100 μM). Left side of the panel: migration of protein standards with mass kilodaltons indicated.

FIG. 4. Degradation of THV proteins is via the ubiquitin-proteasome pathway. HeLa- 16 cells were electroporated with pTV or pTHV plasmids, split into aliquots and treated 24 hours with ou without MG132 (2.5 μM). Cells were analysed for luciferase activities (FIG. 4A). One representative experiment out of three is shown. Cell extracts were performed for wertern blot analysis asz decribed in FIG. 2B (FIG. 4B). C: control cells electroporated with a control plasmid. —: normoxia 21% O2; D: doxycycline (1 μg/ml); Def: desferrioxamine (130 μM); DP: 2-2′-dipyridyl (100 μM). Left side of the panel: migration of protein standards with mass kilodaltons indicated.

FIG. 5. In vivo hypoxic induction of AAT secretion in the serum of mice injected with AAV vectors. Group B is a group of six mice injected with 9.6×10e7 i.p. of AAV-tetO-CMV-AAT. Group C is a group of six mice injected with 4×10e7 i.p. of AAV-tetO-CMV-AAT and 8×10e8 i.p. of AAV-CMV-TV. Mice 1 and 2 were injected with 4×10e7 i.p. of AAV-tetO-CMV-AAT and 8×10e8 or 2×10e9 i.p. of AAV-CMV-THV 401-658, respectively. Mouse 3 was injected with 4×10e7 i.p. of AAV-tetO-CMV-AAT and 8×10e8 i.p. of AAV-CMV-THV 530-603. Mouse 4 was injected with 3×10e7 i.p. of AAV-tetO-CMV-AAT and 6×10e8 i.p. of AAV-V-CMV-THV 530-658. Blood samples were taken and serum were analysed by Elisa for hAAT protein levels.

FIG. 6. Nucleotide and amino acid sequences. In the nucleotide sequences, the first and the last codons coding for the first and the last amino acid, respectively, of a given HIF1-alpha fragment are underlined. In the amino acid sequences, the corresponding first and last amino are circled. (A) nucleic acid encoding THV 401-603; (B) nucleic acid encoding THV 401-658; (C) nucleic acid encoding THV 530-603; (D) nucleic acid sequence encoding THV 530-658; (E) nucleic acid sequence encoding THV 530-826; (F) amino acid sequence of THV 401-603; (G) amino acid sequence of THV 401-658; (H) amino acid sequence of THV 530-603; (I) amino acid sequence of THV 530-658; (J) amino acid sequence of THV 530-826.

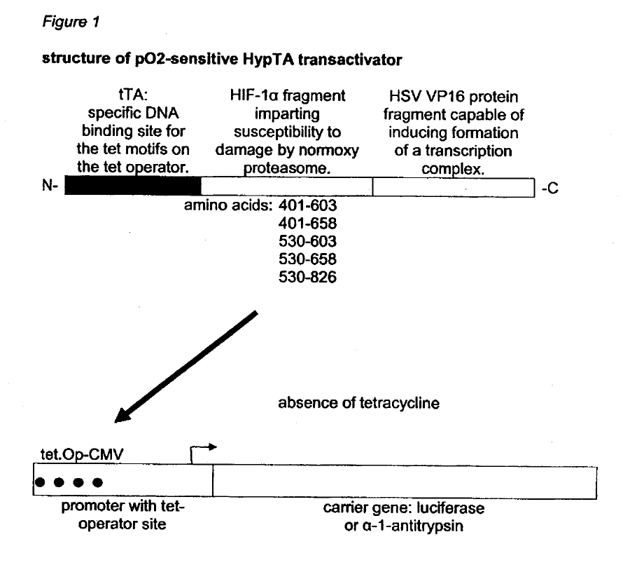

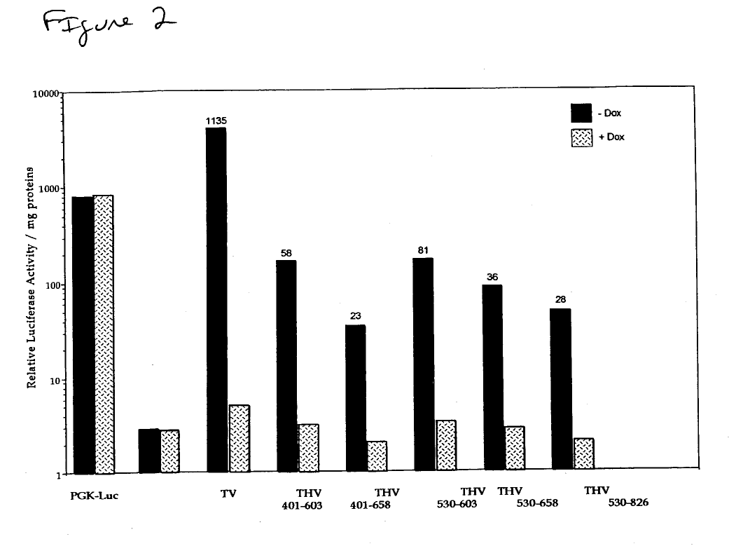

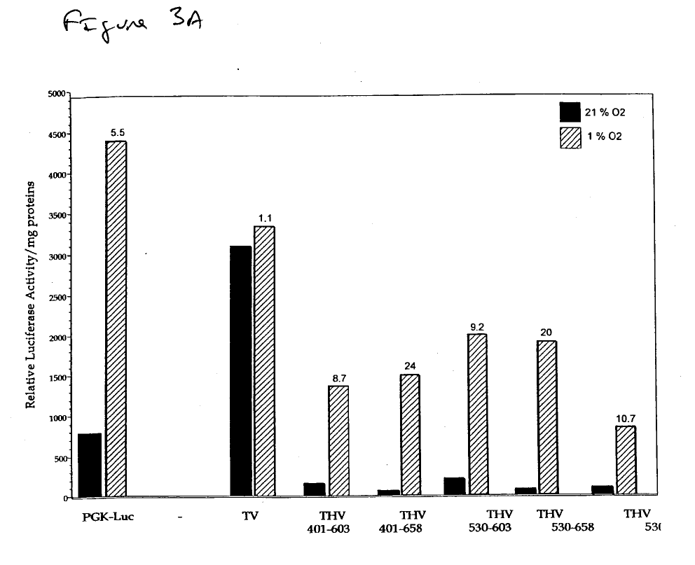

### DETAILED DESCRIPTION OF THE INVENTION

The present invention provides an isolated polynucleotide which codes for a domain of a transcription factor, where the domain confers to the transcription factor susceptibility to degredation under normoxia conditions. The polynucleotide may be DNA or RNA, and may be single or double stranded.

In this system the introduction of only one domain of HIF1-α in the transactivator tTA allows both the degradation by the proteasome of the transactivator under normoxia and transactivation of the tetO-CMV promoter under hypoxia. The Inventors showed that the minimal domain implicated in the inhibition of transactivation of the tetO-CMV promoter under normoxia was between amino acids 530 and 603 of HIF1-α. They showed by western blot that it was specifically the HIF1-α motivate that was responsible for the instability of the protein under normoxia and that the proteasome was implicated in the degradation process. Under hypoxic conditions the protein was not degradated and the transactivation occurred.

The Inventors demonstrate that the stability of the chimeric transactivator is related to the degradation by the proteasome under normoxia and that the regulation in vivo was related to the partial oxygen pressure. Since mice were placed into a chamber filled continuously with 7% oxygen, accessing that the regulation was due to oxygen pressure. Particularly, in these studies mice were placed in a hypoxic chamber with 7% oxygen and the inventors observed a specific induction of the system, thus making the proof that the regulation was due to the partial oxygen pressure.

In one embodiment, the polynucleotide codes for a domain of HIF1-alpha. In a preferred embodiment, the domain comprises amino acids between residues 530 to 603 of the HIF1-alpha, i.e., a plurality of contiguous amino acids within that sequence. In another preferred embodiment, the domain comprises amino acids 530 to 603 of the HIF1-alpha. In a further embodiment, the domain is amino acids 530 to 603 of the HIF1-alpha.

The present invention also provides a chimeric transactivator comprising a domain of a transcription factor, where the domain confers to the transcription factor susceptibility to degredation under normoxia conditions. Thus, the chimera contains a transactivator.

In a preferred embodiment, the transactivator comprises the tetracycline transactivator protein. In another embodiment, the transcription factor is HIF1-alpha, as described above.

The present invention also provides an isolated polynucleotide which codes for the chimeric transactivator described above. The polynucleotide may be DNA or RNA, and may be single or double stranded.

In one embodiment, the chimeric transactivator comprises the tetracycline transactivator protein. In another embodiment, the transcription factor is HIF1-alpha, as described above.

The present invention also provides a vector comprising the polynucleotide described above. In a preferred embodiment, the vector may be a plasmid.

The present invention also provides a composition, comprising:

(a) the polynucleotide described above and

(b) a polynucleotide which contains a sequence that codes for a target gene and a promoter which is regulated by the chimeric tranactivator coded for by (a).

In one embodiment, the chimeric transactivator comprises the tetracycline transactivator protein. In another embodiment, the transcription factor is HIF1-alpha, described above. In these composition, the polynucleotides (a) and (b) may be incorporated into a vector.

In a preferred embodiment, the target gene is erythropoietin. In another preferred embodiment, the target gene is VEGF.

The present invention also relates to a method of expressing a target gene in a subject, comprising administering the composition described above to the subject.

In one embodiment, the subject is a mammal. In a preferred embodiment, the subject is a human.

In one embodiment, the target gene is erythropoietin. In another preferred embodiment, the target gene is VEGF.

The present invention also provides a method of increasing the number of blood vessels in a subject, comprising administering an effective amount of the composition of described above to the subject.

In one embodiment, the subject is a mammal. In a preferred embodiment, the subject is a human.

Dosage levels and methods of administering the inventive compositions may be readily determined by those skilled in the art.

### EXAMPLES

Having generally described this invention, a further understanding can be obtained by reference to certain specific examples which are provided herein for purposes of illustration only and are not intended to be limiting unless otherwise specified.

Material and Methods

Animals

C57B16 mice between 3-4 months old were injected with AAV vectors. Mice were anesthetised with Avertine and 100 μl vector was injected per muscle. Depending on the titers of each AAV vector, either one or two tibialis anterior were injected, and one or two hind limb muscles. Blood samples were taken by orbital puncture, serum was isolated by centrifugation and analysed for AAT expression. For hypoxic conditions, mice were placed in a hermetic chamber with 7% oxygen, 93% azote. Hypoxia was checked by measuring erythropoietin concentration in serum. Detection was performed by Elisa with the commercial R and D kit.

Constructions

PcDNA3 (Clontech) was used as a backbone plasmid to express chimeric constructs under the control of the CMV promoter.

PUHD15.1 (CMV-tTA) and pUHD13.3 (tetO-CMV-luciferase) were kindly provided by Dr Hermann Bujard (Clontech). 621 bp of TetR (T) and 386 bp of VP16 (V) fragments were isolated by PCR from pUHD15.1 with primers: 5′-GGCCCCAAGCTTGG GCATGTCTAGATTAGAT-3′ and 5′-GTACTCCGGATCCGGACCCACTTTCACATTT-3′ for tetR, and 5′-GATAGCTCGAGGCGTACAGCCGCGCGCGTA-3′ and 5′GCGGCTCTAGACTACCCACCGTACTCGTC-3′ for VP16.

The plasmid coding for murine HIF1-alpha (H) was kindky provided by Dr. Roland H. Wenger. Different fragments were isolated by PCR. Motives corresponding to the amino acids 401 to 603 (primers pr-401 and pr-603), 401 to 658 (pr-401 and pr-658), 530 to 603 (pr530 and pr-603), 530 to 658 (pr-530 and pr-658) and 530 to 826 (pr-530 and pr-826) of HIF1-alpha. The chimeric constructs were named THV.

Primers sequences were:

| | |
|---|---|
| pr-401: | 5′-GTATTCCGGATCCGCTCCAGCTGCCGGCGA-3′ |
| pr-603: | 5′-GTCCGGTCGACCTGGAACCCAGTAACTGTGC-3′. |
| pr-658: | 5′-GTCCGGTCGACGTATGCTGATGCCTTAGC-3′. |
| pr-530 | 5′-GTATTCCGGATCCTTCAAGTTGGAACTGGTGGAA-3′ |
| pr-826 | 5′-GTCCGTCGACGTTAACTTGATCCAAAGCTCT-3′. |

All fragments named T, H and V were subcloned in the pCRII cloning kit. The T fragment had a Hind III site in 5′ and BamHI site in 3′, the H fragment had a BamHI site in 5′ and SalI site in 3′, and the V fragment had a XhoI site in 5′ and XbaI site in 3′. The 3 fragments were cloned in frame into the pcDNA3 backbone opened in its muliple cloning sites: Hind III and XbaI. The resulting plasmid was named pTHV.

Adeno-Associated vectors (AAV-2) were constructed by inserting the different cassettes into the AAV-2 backbone (pSUB201 (28). The cDNA for human AAT was kindly provided by Kathy Ponder.

The hAAT cDNA was cloned in the pUHD13.3 to produce ptetO-CMV-AAT. The cassette containing the hAAT cDNA under the control of the tetO-CMV promoter and the polyadenylation site of SV40 was then cloned into pSUB 201 in SnaBI sites. The resulting plasmid was named AAV-tetO-CMV-AAT.

To construct the AAV-CMV-tTA plasmid, the fragment containing the tTA sequence under the control of CMV promoter and with the polyadenylation signal of SV40 from pUHD15.1 was inserted in pSUB201 in SnaBI sites.

For each AAV-CMV-THV, the CMV-THV fragment of pTHV was cut by NruI and XbaI enzymes and the fragment was cloned into an AAV vector that already contains the polyadenylation signal of SV40.

Viral preparations were made in the laboratory of Philippe Moullier in Nantes via the GVPN (Gene Vector Production Network) of the AFM (Association Francaise contre les Myopathies).

Electroporation of HeLa Cells

HeLa cells were maintained in Dulbecco Modified Medium with Glutamax (Gibco) supplemented with 10% fetal calf serum. For transfection, 5×10e6 HeLa cells were electroporated with a Gene Pulser (Biorad) at 200 V, 960 microfarads in the presence of 25 μg of each plasmid, NaCl 3M in 50 microliters. For control experiments a Bluescript pBSK+ was electroporated instead of the plasmid of interest. Each suspension of cells was then split in aliquots and incubated either in medium alone or in medium supplemented with doxycycline (1 μg/ml), cobaltous chloride (100 μM), desferrioxamine (Def) (130 μM), 2-2′-dipyridyl (DP) (100 μM) or the MG132 proteasome inhibiteur (2.5 μM). For hypoxic conditions cells were placed at 37° C. in a closed chamber with 1% oxygen, 5% CO2 and 94% N2 (Air Liquide).

Luciferase Dosages

Electroporated cells were trypsinized and the pellet was lysed in 200 μl LB buffer (Tris Phosphate pH 7.8 25 mM, MgCl2 8 mM, dithiothreitol 1 mM, Triton×100 1%, glycerol 15%). The lysat was then centrifugated at 4° C., the supernatant was collected and luciferase activity was measured using a luminometer (Lumat LB 9501, Berthold). Results were expressed as relative luciferase activity per mg of cell lysates protein. Protein concentration determination was performed with the BCA Protein Assay Kit (Pierce).

Human Alpha-Anti-Trypsine (hAAT) Detection by Elisa

Plates of 96 wells (Maxisorp Immunoplate, Nunc) were coated overnight at 4° C. with a goat anti-AAT antibody (Incstar) in a sodium carbonate buffer 0.1 M. The plate was saturated overnight at 4° C. with TBS-BSA 2% (TBS: Tris 0.05 M, NaCl 0.1 M). Supernatants, serum samples or known amounts of hAAT were then incubated overnight at 4° C. Revelation was performed with a rabbit anti-AAT antibody (1/1000; DAKO A0012) at 37° C. for one hour, followed by a pork biotinylated anti-rabbit antibody (1/10000; DAKO E0431), and streptavidin (1/10000; DAKO P0397). The reaction was developed with OPD (DAKO) and then stopped with sulfuric acid. The optic densities were read at 450 nm.

Western Blot

Cells were trypsinised, washed and sonicated 30 secondes at 18 watts at 4° C. After centrifugation 10 minutes at 4° C., the supernatant was mixed with Laemmli sample buffer, denaturated 5 minutes at 95° C. prior to SDS-polyacrylamide gel electrophoresis. After transfer by electrophoresis at 25 mA overnight, the membrane (Hybond C-super, Amersham) was blocked using Phosphate Buffer Saline supplemented with 5% dry milk powder and NP40 1% prior to immunostaining. Proteins were labeled with an antibody directed againt the VP16 part of the protein (Clontech, 1/200) followed by peroxidase-conjugated anti-rabbit immunoglobulin. Peroxidase activity was detected by enhanced chemiluminescence (ECL-Plus, Amersham).

Results and Discussion

Constructions of Chimeric Proteins Based on the Tetracycline Regulated System Originally Described by the Group of Hermann Bujard.

To identify HIF1-alpha amino acid sequences that could mediate oxygen-dependent transcriptional activation, plasmids were constructed encoding the DNA-binding domain of tetR fused in frame both with HIF1-alpha coding sequences and the C-terminal transactivation domain of VP16. Choosen HIF1-alpha amino acid sequences ranged from amino acid 401 to 603, 401 to 658, 530 to 603, 530 to 658 and 530 to 826. Chimeric constructs were placed under the control of the CMV promoter and were named pTHV. The control plasmid pUHD 15.1 (20) expressed the tetR-VP 16 (tTA) protein without any HIF1-alpha motive (pTV).

The reporter plasmid encoded for a luciferase gene inserted downstream of a minimal CMV promoter that contains seven binding sites for the E.Coli tetracycline repressor (tetO-CMV)(20). HeLa cells were stably transfected with this vector and one clone named HeLa-16 was used in all transient transfection assays.

HeLa-16 cells were electroporated with each pTHV or pTV. After electroporation cells were split into aliquots, and exposed in parallel to 21% O2, 1% O2, 2,2′-dipyridyl (DP), desferrioxamine (Def), cobaltous chloride (Co) or MG132. Luciferase activities were measured 24 hours later and normalised as RLU (relative light unit) per mg of proteins.

Localization of HIF1-Alpha Amino-Terminal Domains that Confer Oxygen-Dependent Instability to Chimeric Proteins Under Normoxia.

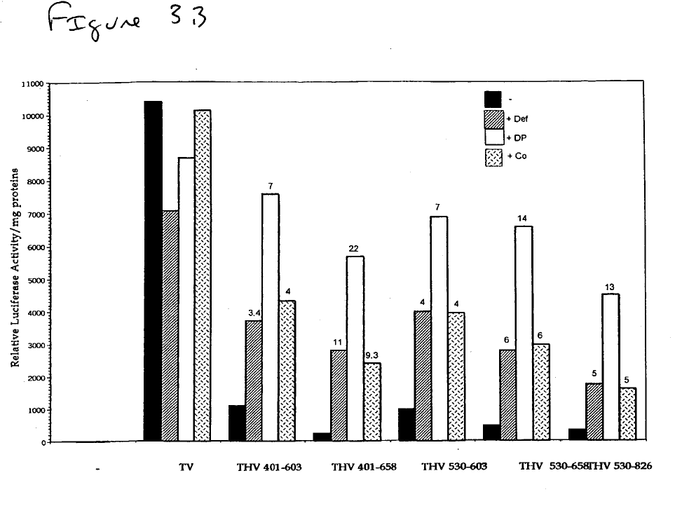

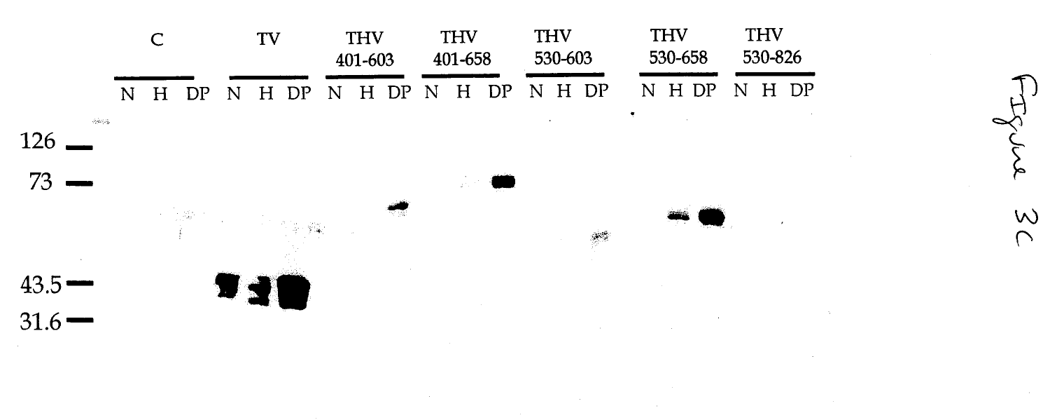

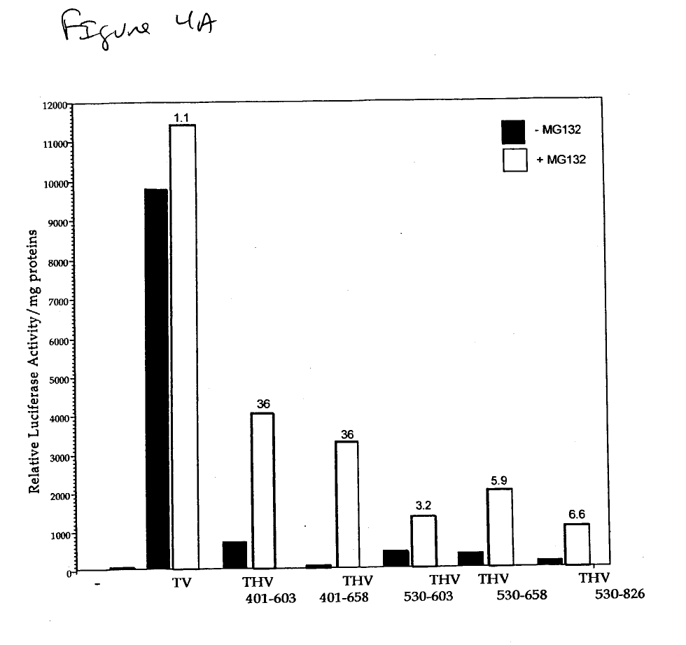

The transactivation levels of the tetO-CMV promoter were analyzed by the different THV chimeric proteins under normoxia (FIG. 2). Background levels of luciferase activity in HeLa-16 cells was between 2.7 to 5.2 RLU/mg proteins (2.7+3+5.2). As expected in the presence of ptTA, the tetO-CMV promoter of HeLa-16 cells was transactivated and luciferase activity measured was up to 1135 fold higher than in control cells. On the contrary when the pTHV plasmids were electroporated, the transcriptional induction levels were very low ranging from 23 to 81 fold. These datas indicate that the introduction of any of the 5 HIF1-alpha motives (amino acids 401 to 603, 401 to 658, 530 to 603, 530 to 658 and 530 to 826) into the tTA protein reduce the transactivation activity of the fusion proteins under normoxia. The minimal domain of HIF1-alpha sufficient to reduce highly significantly the induction of the tetO-CMV promoter by tTA was comprised between amino acids 530 and 603. Two other domains of HIF1-alpha could also be identified in this experiment: amino acids 401 to 530 and 603 to 658. When the smallest motive of HIF1-alpha 530-603 was present on pTHV the background luciferase level was the highest one (81 fold) among all pTHV. When either the adjacent domain 401-530 or 603-658 was added to 530-603, background levels were significantly lower (58 and 36 folds, respectively), suggesting that these two domains were additional targets for degradation under normoxia. This was confirmed with the pTHV401-658 protein containing both domains as it was the one showing the lowest background level (23 fold).

Regulation by Tetracycline of the Fusion Proteins.

As the THV fusion proteins contained the tetR DNA-binding domain of the E. Coli tetracycline operon, we asked whether the regulation by tetracycline was maintained in the chimeric proteins. Electroporated HeLa-16 cells were then incubated in the presence or absence of doxyxycline. As shown in FIG. 2, the transcription from the tetO-CMV promoter was completely repressed in the presence of the drug with each pTHV protein. These datas indicated that (i) the DNA-binding domain of the fusion protein was still able to bind tetracycline and (ii) this binding fully prevents the transactivation of the tetO-CMV promoter.

Localization of the HIF1-Alpha Amino-Terminal Transactivation Domains Under Hypoxia.

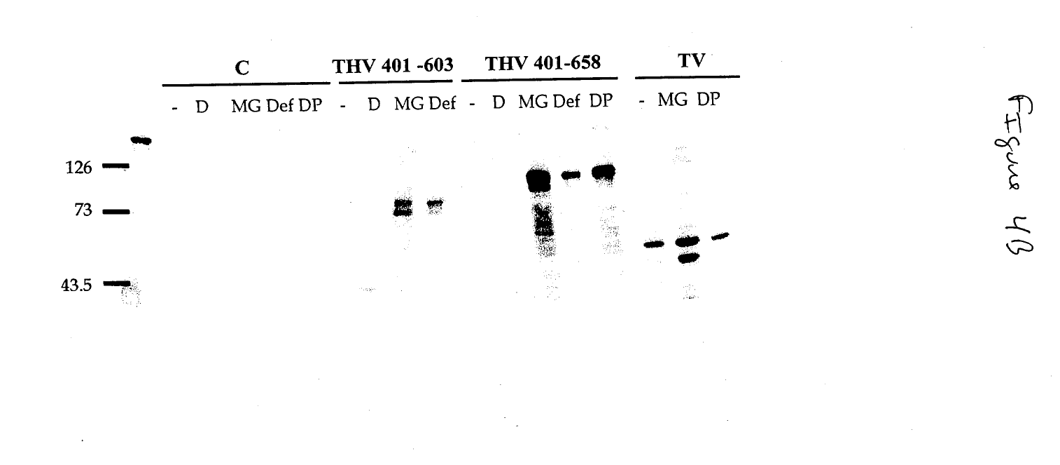

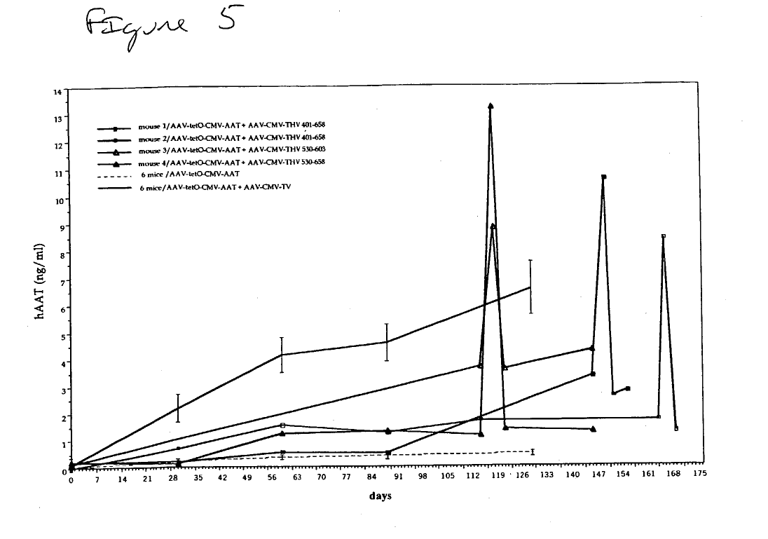

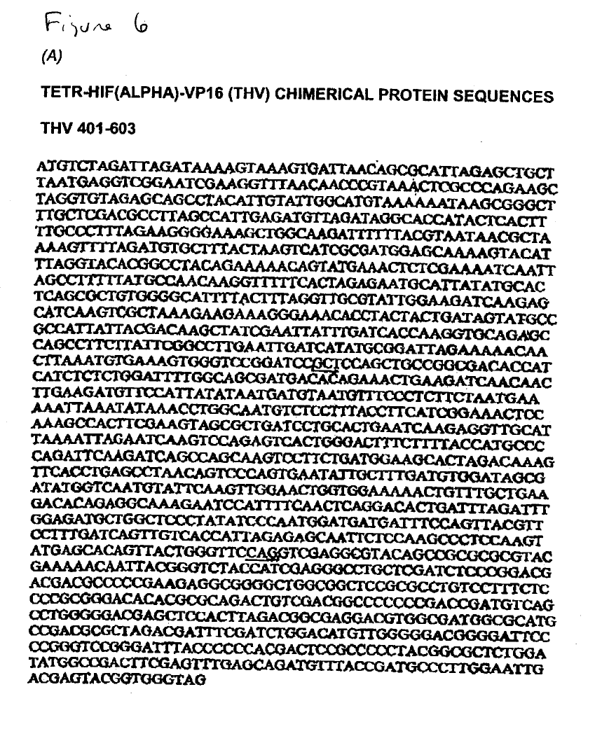

Electroporated cells were incubated 24 hours either under normoxia (21% O2) or under hypoxia (1% O2) (FIG. 3A). As a positive control a plasmid encoding for the luciferase reporter gene under the control of the hypoxia inducible PGK promoter was used. As expected under hypoxia the PGK promoter was transactivated and luciferase expression increased 5.5 fold. When pTV was electroporated no hypoxic transactivation of the tetO-CMV promoter was observed, as no HIF1-alpha motive was expressed. On the contrary when pTHV plasmids were electroporated luciferase activities were specifically induced under 1% O2. Inductions were ranging from 8.7 to 24 fold. The maximum induction level of pTHV was two to three times lower than the one with pTV. One positive and one negative domain of HIF1-alpha could be identified. With the 401-603 or 530-603 domains of HIF1-alpha, induction levels were in the range of 10 under hypoxia. However when the domain between 603 and 658 was added, the induction ratio was increased twice, suggesting that this domain had a positive effect on transactivation. On the other hand, the domain between 658 and 826 of HIF1-alpha seemed to binds negative factors. In conclusion, these data indicated that (i) in the context of a fusion protein the hypoxic transcriptional activation function of HIF1-alpha sequences were maintained and (ii) the minimal motive of HIF1-alpha that prevents degradation under hypoxia and subsequently stimulates transcription was between amino acids 530 and 658.

Transcriptional Activation Function of HIF1-Alpha Stimulated by Other Inducers of the Hypoxia Signal Transduction Pathway.

It has previously been demonstrated that the HIF1-alpha transactivation domain function was induced in cells exposed to desferrioxamine (Def), cobaltous chloride (Co) or 2-2′-dipyridyl (DP). To determine if this property was conserved in the context of the chimeric THV proteins, HeLa-16 cells were electroporated with pTHV plasmids and incubated 24 hours with those different chemicals (FIG. 3B). When pTV was electroporated, the addition of drugs had no significant effect on the transactivation of the tetO-CMV promoter. In the presence of pTHV the addition of each of the 3 chemicals mimiced the hypoxic state. The most effective chemical was DP with induction ratios similar to those measured under 1% O2. Moreover wth this chemical the transcriptional activity of the THV fusion proteins was in the range of the control pTV transactivator. With cobalt and desferrioxamine the transactivation levels were lower than with DP. The best induction folds were measured with the THV protein containing the fragment 401-658, as it is the one showing the lowest background under normoxia. These results indicate that all HIF1-alpha motives inserted into tTA can be stimulated by inducers of the hypoxia signal transduction pathway.

To confirm these datas an immunoblot analysis of electroporated HeLa-16 cells was performed. As shown on FIG. 3C, the expression level of the TV protein was the same under 21% O2, 1% O2 or with DP. On the contrary, with the THV constructs no signal was detected under normoxia whereas under both conditions of hypoxia (1% O2 and DP) the THV protein levels were greatly induced. The induction was more pronounced with DP. These results indicate that the HIF1-alpha sequences of THV proteins contain motives implicated in the regulation of the stability of the protein under hypoxia.

Inhibition of the 26S Proteasome Activity Leads to Accumulation of Fusion Proteins Under Normoxia.

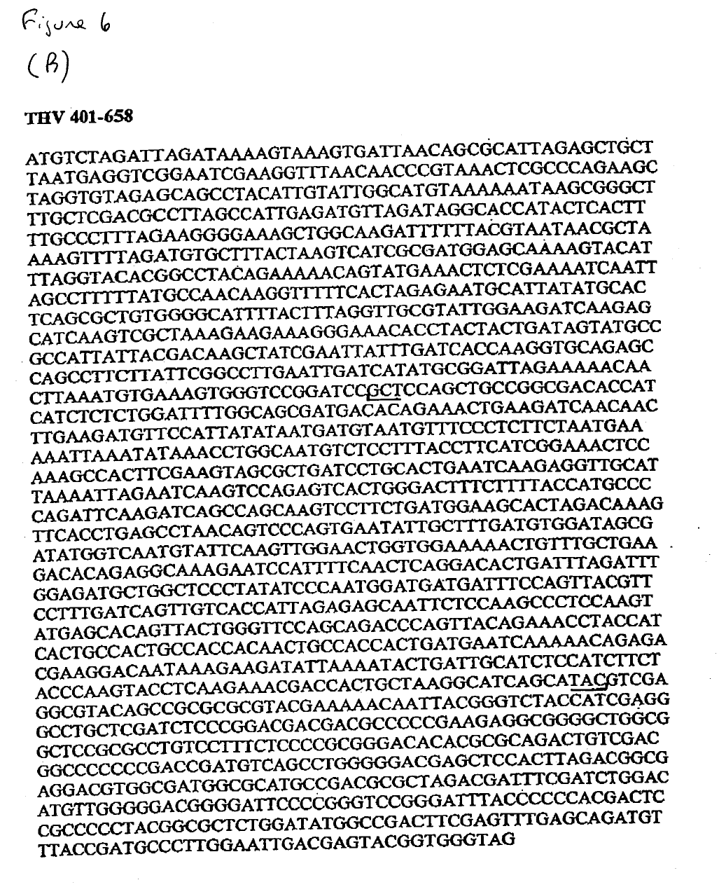

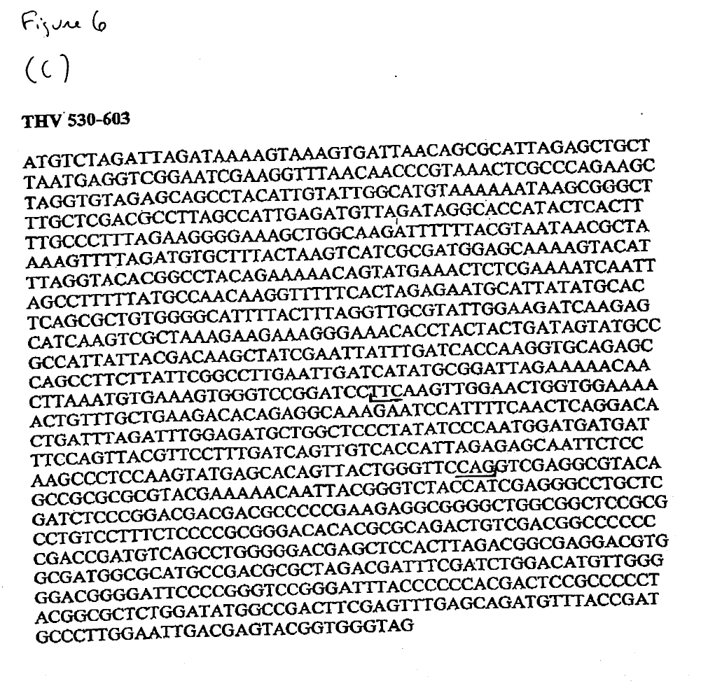

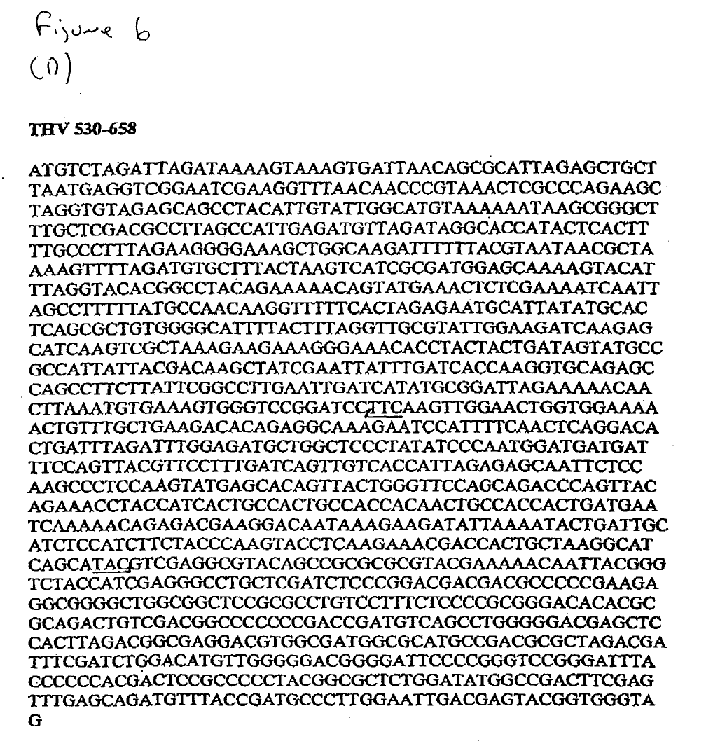

Electroporated HeLa-16 cells were treated with MG132 to analyse the degradation of the THV proteins by the ubiquitin-proteasome pathway under normoxia (FIG. 4A). With the TV transactivator no significant induction of the tetO-CMV promoter was measured in the presence of MG132. With the THV chimeric proteins luciferase activities increased significantly when cells were incubated in the presence of MG132. These results show that the HIF1-alpha fragments conferred susceptibility for degradation by the proteasome to the chimeric transactivator in condition of normoxia. The fragment containing the amino acids 530 to 603 seemed to contain the minimum motives necessary for the degradation under normoxia. On the other hand the fragment of HIF1-alpha containing the amino acids 401 to 603 seemed to contain several motives implicated in the proteasome degradation as in the presence of MG132 the induction level was in the range of 36 fold, which is ten times more than the level of induction with the 530-603 fragment. This suggest that the minimum oxygen-dependent degradation domain within HIF1-alpha which controls the degradation of THV by the ubiquitin-proteasome pathway is between 530 and 603.

An immunoblot was performed to analyse the levels of the THV proteins in the presence of MG132 (FIG. 4C). The protein level of TV was not different with ou without treatment with MG132, or even with DP. The protein levels of the two chimeric THV constructs presented on the Figure, THV 401-603 and THV 401-658, were greatly induced in the presence of MG132, as in the presence of Def or DP. These datas show that when the ubiquitin-proteasome pathway was blocked with MG132 THV proteins were stabilised and could transactivate of the tetO-CMV promoter.

In Vivo AAV Gene Transfer.

One objective was to obtain an oxygen-dependent expression of a reporter protein in vivo. The choosen reporter protein was the human alpha-1 anti-trypsin (AAT), which is easily detectable in serum and is not immunogenic in the C57b1/6 mouse strain. AAV-2 vectors were constructed. The reporter AAV vector contains the AAT cDNA inserted downstream of the minimal tetO-CMV promoter. The transactivating AAV vectors contain either TV or THV expression cassettes, with the strong CMV promoter. A control vector expressed the hAAT cDNA under the control of the CMV promoter.

Different groups of normal C57B16 mice were injected intramuscularly with the AAV vectors. In the group A, six mice were injected with the AAV-CMV-AAT vector: 2×10e7 infectious particules (i.p.) were injected intramuscularly. AAT secretion in the serum of mice was followed over several weeks. Secretion at day 0 was 0.476+/−0.12 ng/ml and increased progressively reaching hAAT secretion levels in the range of 20 to 40 hAAT ng/ml after 132 days (not shown).

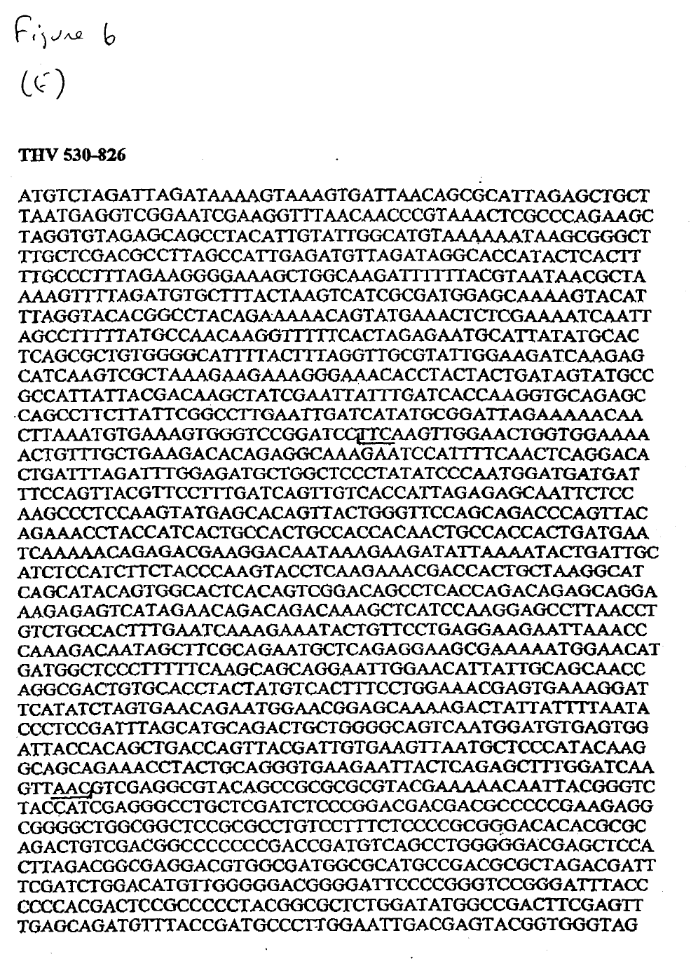

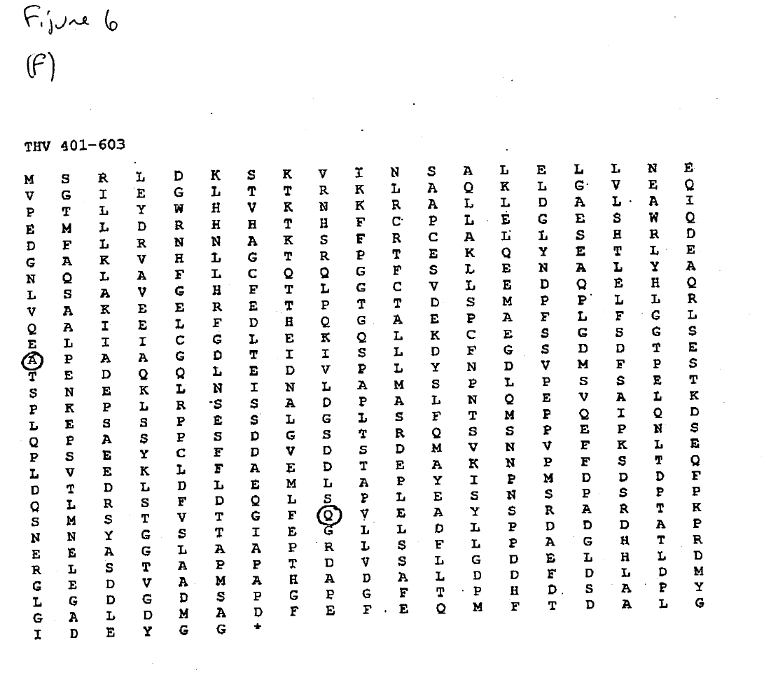

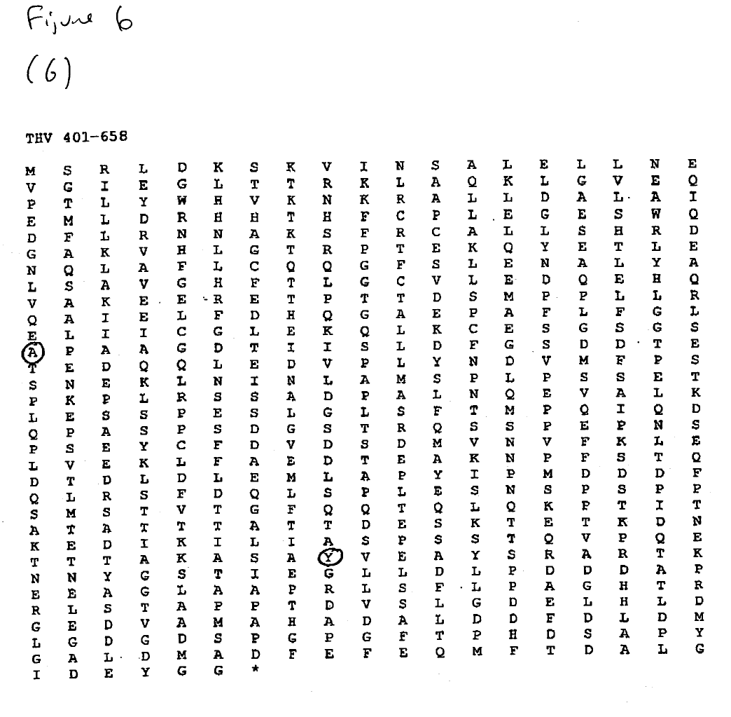

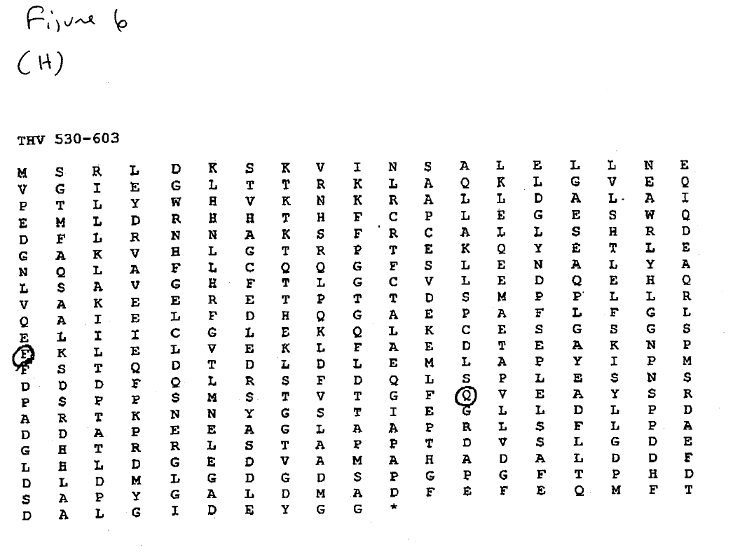

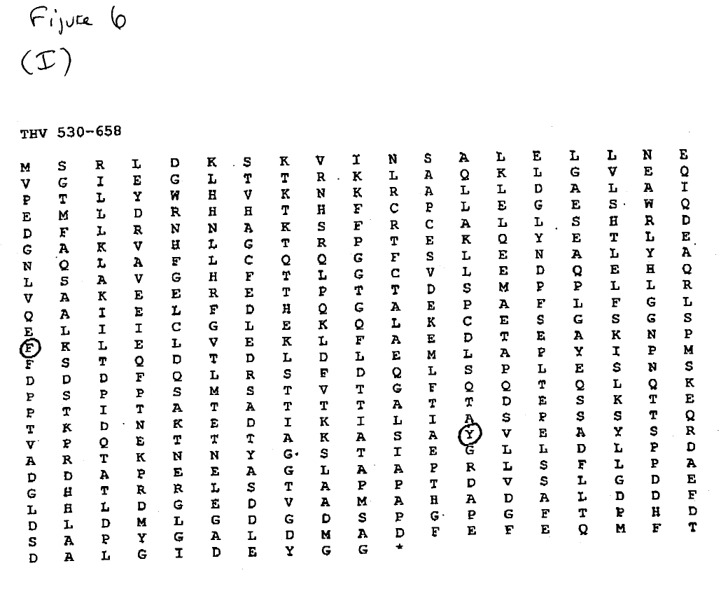

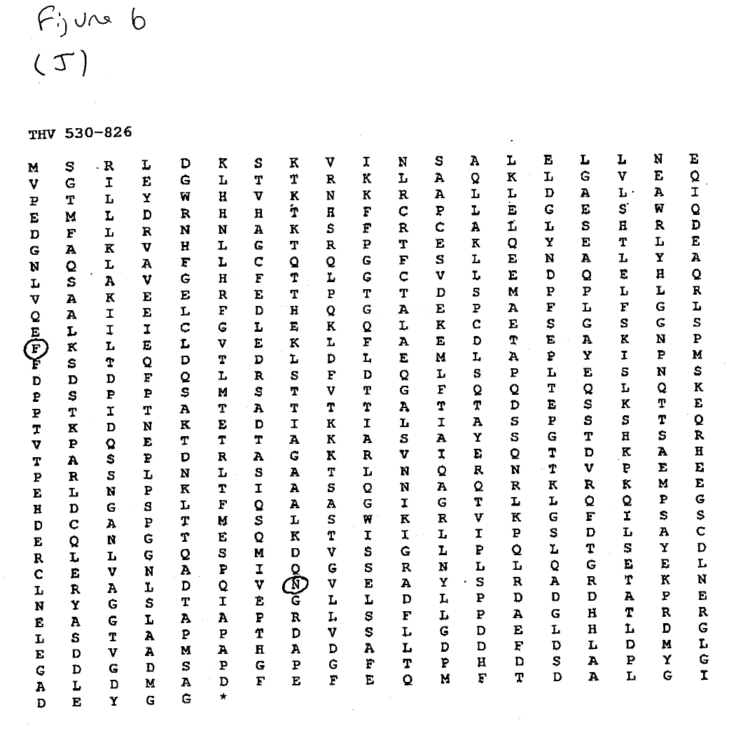

One group of six mice were injected with the AAV-tetO-CMV-AAT vector alone (group B). As expected hAAT expression from the minimal tetO-CMV promoter remained very low. The secretion level increased from 0.215+/−0.169 hAAT ng/ml at day 0 to 0.458+/−0.098 hAAT ng/ml after 132 days (FIG. 5).

In group C six mice were injected with both AAV-tetO-CMV-AAT and AAV-CMV-TV. As shown in FIG. 5, the tetO-CMV promoter was induced by the TV transactivator and hAAT secretion increased from 0.108+/−0.054 hAAT ng/ml at day 0 to 6.54+/−0.98 hAAT ng/ml at day 132. The secretion level reached with the TV protein was 3 to 6 times lower than with the CMV promoter.

Several groups of mice were injected with both AAV-tetO-CMV-AAT and AAV-CMV-THV. In those mice background levels of hAAT secretion were in the range of 1 to 3 ng/ml. Compared to group B secretion levels, these results indicate that the tetO-CMV promoter was transactivated by the THV proteins even under normoxia, as shown in vitro. Nevertheless these background levels were very low compared to group A mice. Three months after injection, a time at which background serum levels of hAAT were stabilized, some mice were maintained at 7% oxygen for 24 hours. In four mice the hAAT secretion levels were significantly induced, followed by a return to background levels after 3 days (FIG. 5).

Mice 1 and 2 were injected with both AAV-tetO-CMV-AAT and AAV-CMV-THV 401-658. In mouse 1 a 3.2 fold increase in hAAT secretion was measured after hypoxia induction. The hAAT level increased from 3.3 ng/ml at day 149 to 10.6 ng/ml 24 hours after hypoxia and returned to 2.6 ng/ml 3 days later. In mouse 2 a 6.4 fold induction ratio was measured. The hAAT level increased to 8.4 ng/ml 24 hours after hypoxia and returned to 1.3 ng/ml 3 days later.

Mouse 3 was injected with both AAV-tetO-CMV-AAT and AAV-CMV-THV 530-603. A 2.4 fold induction ratio was measured. The hAAT level increased from 3.7 ng/ml at day 117 to 8.8 ng/ml after 24 hours under hypoxia and returned to 3.6 ng/ml 3 days later.

Mouse 4 was injected with both AAV-tetO-CMV-AAT and AAV-CMV-THV 530-658. A 11 fold induction ratio was measured. The hAAT level increased from 1.1 ng/ml at day 117 to 13.2 ng/ml 24 hours after hypoxia and returned to 1.4 ng/ml 3 days later.

These results show that in vivo the secretion of a foreign protein can be regulated by a physiological stimulus like oxygen pressure. The minimal domain of HIF1-alpha that conferred this regulation capacity to the TV protein was between amino acids 530 and 603.

### Deposit of Biological Materials

Two plasmids were deposited at the CNCM (Collection Nationale des Cultures de Microorganisms, Institut Pasteur, 28, rue du Dr Roux, 75724 Paris Cedex 15, France) on Apr. 18, 2002:

AAV-CMV-THV 401-658 under accession No. CNCM I-2853

AAV-CMV-THV 530-658 under accession No. I-2854.

Obviously, numerous modifications and variations of the present invention are possible in light of the above teachings. It is therefore to be understood that within the scope of the appended claims, the invention may be practiced otherwise than as specifically described herein.

### References

1. Koury, S. T., Bondurant, M. J. & Koury, M. J. (1988) Blood 71, 524-527.

2. Koury, M. J. & Bondurant, M. C. (1992) Eur. J. Biochem. 210, 649-663.

3. Krautz, S. B. (1991) Blood 77, 419-434.

4. Lee-Huang, S., Lin, J. J., Kung, H., Lira Huang, P., Lee, L. & Lee Huang, P. (1993) Gene 128, 227-236.

5. Beck, I., Ramirez. S., Weinmann, R. & Caro, J. (1991) J. Biol. Chem 266, 15563-15566.

6. Marian, A. & Curtin, P. T. (1993) Proc. Natl. Acad. Sci. USA 90, 3928-3932.

7. Semenza, G. L. & Guam, L. W. (1992) Mol. Cell. Biol. 12, 5447-5454.

8. Pugh, C. W., Tan, C. C., Jones, R. W. & Ratcliffe, P. J. (1991) Proc. Natl. Acad Sci. USA. 88, 10553-10557.

9. Maxwell, P. H., Pugh, C. W. & Ratcliffe, P. J. (1993) Proc. Natl. Acad. Sci. USA. 90, 2423-2427.

10. Wang, G. L. & Semenza, G. L. (1993) J. Biol Chem 268, 21513-21518.

11. Beck L, Weinmann, R. & Caro, J. (1993) Blood 82, 704-711.

12. Rinsch, G., Regulier; E., Deglon, N., Dalle, B., Beuzard, Y. & Aebischer, P. (1997) Human Gene Ther. 8, 1881-1889.

13. Wang, G. L., Jiang, E., Rue, E. A. & Semenza, G. L. (1995) Proc. Natl. Acad Sci. USA. 92, 5510-5514.

14. Jiang, B H, Zheng J Z, Leung S W, Roe R, Semenza G L (1997). J. Biol. Chem. 272, 19253-19260.

15. Pugh, C. W., O'Rourke, J. F., Nagao, M., Gleadle, J. M. & Ratcliffe, P. J. (1997) J. Biol. Chem 272, 11205-11214.

16. O'Rourke, J. F, Tian, Y M, Ratcliffe, P J, Pugh C W. (1999). J. Biol. Chem. 274, 2060-2071.

17. Salceda, S, Caro, 7. (1997). J. Biol. Chem. 272, 22642-22647.

18. Huang, L., Gu, J., Schau, M. & Bunny, H. (1998) Proc. Natl. AcadSci. USA 95, 7987-7992.

19. Srinivas, V, Zhang, L P, Zhu, X H, Caro. J. (1999). Biochem Biophys. Res. Commun. 260; 557-561.

20. Kallia, P., Wilson, W., O'Brien, S., Makino, Y. & Poellinger, L. (1999) J. Biol Chem. 274, 6519-6525.

21. Jaakkola, P, Mole, D R, Tiara, Y, Wilson, M I, Gielbert, J, Gaskell, S J, von Kriegsheim, A, Hebestreit, H F, Mukherji, M, Schofield, C J, Maxwell, P H Pugh, C W, Ratcliffe, P J. Science 2001. 292:468-472.

22. Ivan, Kondo, K. Yang, H. Kim, W. Valiando, J. Ohh, M, Salic, A, Asara, J M, Lane, W S, Kaelin Jr, W G. Science 2001. 292: 464-468.

23. Sutter, C H, Laughner, E, Semenza, G L. (2000), Proc. Natl. Acad Sci. USA 97, 4748-4753.

24. Bohl, D., Naffakh, N. & Heard, J. M. (1997) Nature Med 3, 299-312.

25. Bohl, D., Salvetti, A., Moullier, P. & Heard, J. M. (1998) Blood 92, 1512-1517.

26. Rendhal, K. G., Leff, S. E., Otten, G. R., Spratt, S. K., Bohl, D., Van Roey, M, Donahue, B. A., Cohen, L. K., Mandel, R. J., Danos, O. & Snyder, R O. (1998) Nature Biotech. 16, 757-761.17.

27. Gossea, M. & Bujard, H. (1992) Proc. Natl. Acacd, Sci. USA 89, 5547-5551.

28. Samulski, R J., L S., Shenk, T. (1997)., J. Virol. 61, 3096-3101.

29. Payen et al., J. Gene Med., 2001, 3: 498-504.

1. An isolated polynucleotide which codes for a domain of a transcription factor, wherein the domain confers to the transcription factor susceptibility to degredation under normoxia conditions.

2. The polynucleotide of claim 1, which codes for a domain of HIF1-alpha.

3. The polynucleotide of claim 2, wherein the domain comprises amino acids between residues 530 to 603 of the HIF1-alpha.

4. The polynucleotide of claim 2, wherein the domain comprises amino acids 530 to 603 of the HIF1-alpha.

5. The polynucleotide of claim 2, wherein the domain is amino acids 530 to 603 of the HIF1-alpha.

6. A chimeric transactivator comprising a domain of a transcription factor, wherein the domain confers to the transcription factor susceptibility to degradation under normoxia conditions.

7. The transactivator of claim 6, which comprises the tetracycline transactivator protein.

8. The transactivator of claim 7, wherein the transcription factor is HIF1-alpha.

9. The transactivator of claim 8, wherein the domain comprises amino acids between residues 530 to 603 of the HIF1-alpha.

10. The transactivator of claim 8, wherein the domain comprises amino acids 530 to 603 of the HIF1-alpha.

11. The transactivator of claim 8, wherein the domain is amino acids 530 to 603 of the HIF1-alpha.

12. An isolated polynucleotide which codes for the chimeric transactivator of claim 6.

13. The polynucleotide of claim 12, wherein the chimeric transactivator comprises the tetracycline transactivator protein.

14. The polynucleotide of claim 12, wherein the transcription factor is HIF1-alpha.

15. The polynucleotide of claim 14, wherein the domain comprises amino acids between residues 530 to 603 of the HIF1-alpha.

16. The polynucleotide of claim 14, wherein the domain comprises amino acids 530 to 603 of the HIF1-alpha.

17. A vector comprising the polynucleotide of claim 12.

18. The vector of claim 17, wherein the chimeric transactivator comprises the tetracycline transactivator protein.

19. The vector of claim 17, wherein the transcription factor is HIF1-alpha.

20. The vector of claim 19, wherein the domain comprises amino acids between residues 530 to 603 of the HIF1-alpha.

21. The vector of claim 19, wherein the domain comprises amino acids 530 to 603 of the HIF1-alpha.

22. A composition, comprising:
(c) the polynucleotide of claim 12 and
(d) a polynucleotide which contains a sequence that codes for a target gene and a promoter which is regulated by the chimeric tranactivator coded for by (a).

23. The composition of claim 22, wherein the chimeric transactivator comprises the tetracycline transactivator protein.

24. The composition of claim 22, wherein the transcription factor is HIF1-alpha.

25. The composition of claim 24, wherein the domain comprises amino acids between residues 530 to 603 of the HIF1-alpha.

26. The composition of claim 24, wherein the domain comprises amino acids 530 to 603 of the HIF1-alpha.

27. The composition of claim 22, wherein the target gene is erythropoietin.

28. The composition of claim 22, wherein the target gene is VEGF.

29. A method of expressing a target gene in a subject, comprising administering the composition of claim 22 to the subject.

30. The method of claim 29, wherein the subject is a mammal.

31. The method of claim 29, wherein the subject is a human.

32. The composition of claim 29, wherein the target gene is erythropoietin.

33. The composition of claim 29, wherein the target gene is VEGF.

34. A method of expressing a target gene in a subject, comprising administering the composition of claim 23 to the subject.

35. A method of expressing a target gene in a subject, comprising administering the composition of claim 24 to the subject.

36. A method of expressing a target gene in a subject, comprising administering the composition of claim 25 to the subject.

37. A method of expressing a target gene in a subject, comprising administering the composition of claim 26 to the subject.

38. A method of increasing the number of red blood cells in a subject, comprising administering an effective amount of the composition of claim 27 to the subject.

39. The method of claim 38, wherein the subject is a mammal.

40. The method of claim 38, wherein the subject is a human.

41. A method of increasing the number of blood vessels in a subject, comprising administering an effective amount of the composition of claim 28 to the subject.

42. The method of claim 41, wherein the subject is a mammal.

43. The method of claim 41, wherein the subject is a human.
